(19) United States
(12) Patent Application Publication
(10) Pub. No.: US 2015/0133489 A1
(43) Pub. Date: May 14, 2015

(54) COMPACTED MOXIFLOXACIN

(71) Applicant: RATIOPHARM GMBH, Ulm (DE)

(72) Inventors: Dieter Swatschek, Blaubeuren (DE); Max Werner Scheiwe, Maulburg (DE)

(21) Appl. No.: 14/541,274

(22) Filed: Nov. 14, 2014

Related U.S. Application Data
(63) Continuation of application No. 13/132,103, filed on Aug. 8, 2011, now abandoned.

(51) Int. Cl. A61K 9/20; A61K 9/28; A61K 31/4709

(52) U.S. Cl. CPC A61K 9/2095; A61K 31/4709; A61K 9/2054; A61K 9/2018; A61K 9/2009; A61K 9/2013; A61K 9/28; A61K 9/2893
USPC 514/300; 264/115; 427/2.14

(57) ABSTRACT

The invention relates to a process for the preparation of tablets containing moxifloxacin, comprising the steps of (i) providing moxifloxacin, pharmaceutically acceptable salts, solvates or hydrates thereof, optionally mixed with one or more pharmaceutical excipients; (ii) compacting it into a slug; (iii) granulating the slug; and (iv) compressing the resulting granules into tablets; and also tablets, granules and compacted material containing compacted moxifloxacin.

The invention relates to a process for the preparation of tablets containing moxifloxacin, comprising the steps of (i) providing moxifloxacin, pharmaceutically acceptable salts, hydrates or solvates thereof mixed with an adhesive agent; (ii) compacting it into a slug; (iii) granulating the slug; and (iv) compressing the resulting granules into tablets; and tablets, granules and flakes containing compacted moxifloxacin. In addition, the invention relates to tablets containing moxifloxacin with a bimodal pore size distribution.

1-cyclopropyl-7-([S,S]-2,8-diazabicyclo[4.3.0]non-8-yl)-6-fluoro-1,4-dihydro-8-methoxy-4-oxo-3-quinolonic carboxylic acid is known by the INN name “moxifloxacin” and has the following structural formula:

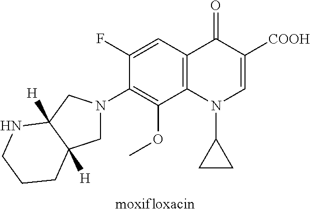 [SMILES: COc1c(N2C[C@@H]3CCCN[C@@H]3C2)c(F)cc2c(=O)c(C(=O)O)cn(C3CC3)c12]

Moxifloxacin is an antibiotic which is used in the treatment of respiratory infections and pneumonia. Moxifloxacin has a bactericidal action which works by inhibiting the bacterial DNA topoisomerases II (DNA gyrase) and IV, which are responsible for the replication and transcription of DNA in the bacterial cell.

The synthesis of moxifloxacin is described in EP 0 350 733 A2, EP 0 757 990 B1, and DE 42 00 414 A1.

In EP 0 780 390 A1, it was found that advantageous pharmaceutical formulations were obtained whenever moxifloxacin was used in the form of the monohydrate. In addition, a process was provided in which crystalline moxifloxacin was not obtained in the form of needles, because needles would become undesirably matted and would impair the pourability of the active agent.

EP 1 017 392 B1 describes moxifloxacin pharmaceutical preparations with controlled release of the active agent. The delayed release was achieved by adding a diffusion lacquer. These formulations were produced by wet granulation in a fluidised bed.

WO 2005/20998 A1 likewise describes moxifloxacin formulations, wherein a waterinsoluble excipient was processed intragranularly and extragranularly by means of wet granulation. The aim of this method of preparation was to provide a bioequivalent formulation to Avelox®.

Finally, in EP 1 128 831 A, it was found that tablets with a high breaking load, or hardness, were only obtained if lactose was used as an excipient and the lactose content of the formulation was between 2.5 and 25%. The use of lactose is often undesirable, however, since a not inconsiderable number of patients suffer from lactose intolerance.

It can therefore be stated that the processes described in the state of the art for the preparation of tablets containing moxifloxacin involve numerous disadvantages, such as in the form of special requirements regarding the lactose content, the crystal structure of the active agent, and the need for special solutions for granulating purposes. In addition, the known tablets are only suitable to a limited extent for lacquer coating. The objective of the present invention is therefore to overcome the disadvantages found in the state of the art. In particular, the aim is to overcome the disadvantages of the state of the art without impairing working safety.

Specifically, it is an object of the invention to provide a process for the preparation of tablets containing moxifloxacin, wherein moxifloxacin can be processed with every known crystal habit (e.g. plates or needles) and/or in any of the known polymorphous forms, i.e. the aim is to enable moxifloxacin to be processed with the active agent in different states. Similarly, the aim is to enable moxifloxacin to be processed with variable water contents. The processing of moxifloxacin with variable water contents is supposed to be made possible above all while achieving advantageous storage stability at the same time.

Specifically, it is an object of the invention to provide tablets which have both a rapid disintegration time (less than 15 minutes, preferably less than 10 minutes) and also the most advantageous breaking strength (more than 160 Newton, preferably more than 180 Newton). In addition, the resulting tablets are supposed to exhibit little abrasion.

In particular, it is an object of the invention to provide a process for the preparation of tablets containing moxifloxacin which exhibit advantageous lacquer coatability. During lacquer coating of the tablets of the invention, it is intended that no spalling should occur.

The intention is likewise to provide a granule formulation of moxifloxacin which can advantageously be used in the production of a suspension to be swallowed. The granules should flow well, not separate during storage, and enable exact dosaging from single-dose and multi-dose containers.

Finally, it is an object of the invention to provide pharmaceutical dosage forms of moxifloxacin which exhibit advantageous storage stability. Similarly, it is intended to ensure an advantageous content uniformity. All the objects mentioned above are supposed to be achieved in particular for a high content of active agent (drug load). In addition, it is intended to achieve the objects while avoiding lactose as an excipient.

The inventors have now unexpectedly found that the objects can be achieved by compacting a mixture of moxifloxacin and adhesive agent into a slug.

The subject matter of the invention is thus compacted material containing moxifloxacin, obtainable by a process comprising the steps of:
(i) providing moxifloxacin or pharmaceutically acceptable salts thereof mixed with an adhesive agent; and
(ii) compacting it into a slug.

The subject matter of the invention is also a process for the preparation of tablets containing moxifloxacin, comprising the steps of
(i) providing moxifloxacin or pharmaceutically acceptable salts thereof mixed with an adhesive agent;
(ii) compacting it into a slug;
(iii) granulating the slug; and
(iv) compressing the resulting granules into tablets, optionally with the addition of further pharmaceutical excipients.

The tablets produced with the process of the invention may optionally be film-coated in a further, optional step (v).

The subject matter of this invention is also tablets and film-coated tablets obtainable by the process of the invention.

A further subject matter of the invention is thus granules, for example for filling in sachets or capsules, containing moxifloxacin, obtainable by a process comprising the steps of:
(i) providing the moxifloxacin or pharmaceutically acceptable salts thereof mixed with an adhesive agent;
(ii) compacting it into a slug; and
(iii) granulating the slug.

During or preferably after step (iii), further excipients may optionally be added to the granules. In particular, excipients to improve flowability, sticking tendency, disintegration characteristics, taste and/or wettability are used for this purpose.

The resulting granules are preferably used for producing a suspension for swallowing. They are preferably filled in suitable packaging. Examples of packaging are capsules, bottles, boxes or preferably sachets. In the case of bottles or boxes, these may contain one daily dose. Alternatively, multiple daily doses, e.g. 10 daily doses, may be filled in bottles or boxes.

Finally, one subject matter of the invention is the use of dry-compacted moxifloxacin for the oral treatment of infections, especially infections of the airways and soft-tissue infections.

The process of the invention for the preparation of tablets containing moxifloxacin is explained in detail in the following paragraphs.

In step (i) of the process of the invention, moxifloxacin is first prepared.

As a matter of principle, the term “moxifloxacin” in the context of this application comprises both moxifloxacin in the form of the free base and also pharmaceutically acceptable salts thereof. These may be one or more salts, which may also be present in a mixture. In addition, the term “moxifloxacin” also comprises possible hydrates or solvates.

The salts used are preferably acid addition salts. Examples of suitable salts are hydrochlorides, carbonates, hydrogen carbonates, acetates, lactates, butyrates, propionates, sulphates, citrates, tartrates, nitrates, sulphonates, oxalates and/or succinates.

In the context of this invention, moxifloxacin is preferably used in the form of the free base or as moxifloxacin hydrochloride.

The moxifloxacin used may contain water. It normally comprises 0.1 to 5% by weight water, preferably 0.2 to 2% by weight water, based on the total weight of the active agent.

In step (i) of the process of the invention, moxifloxacin is mixed with one or more adhesive agent(s).

“Adhesive agents” generally means agents which improve the adhesive characteristics of the resulting compacted material. In addition, adhesive agents are preferably characterised by the fact that they increase the plasticity of the tableting mixture, so that solid tablets form during compression.

In one possible embodiment, the adhesive agent is a polymer. In addition, the term “adhesive agent” also includes substances which behave like polymers. Examples of these are fats and waxes. Furthermore, the adhesive agents also include solid, non-polymeric compounds which preferably contain polar side groups. Examples of these are sugar alcohols or disaccharides.

In addition, the polymers which can be used as adhesive agents preferably have a number-average molecular weight of 1,000 to 500,000 g/mol, more preferably 2,000 to 90,000 g/mol. When the polymer used in the preparation of the intermediate is dissolved in water in an amount of 2% by weight, the resulting solution preferably has a viscosity of 0.1 to 8 mPa/s, more preferably 0.3 to 7 mPa/s, especially 0.5 to 4 mPa/s, measured at 25° C. The friability is preferably determined in accordance with Ph. Eur. 6.0, section 2.2.10.

Hydrophilic polymers are preferably used for the preparation of the intermediate. This refers to polymers which possess hydrophilic groups. Examples of suitable hydrophilic groups are hydroxy, alkoxy, acrylate, methacrylate, sulphonate, carboxylate and quaternary ammonium groups.

The intermediate of the invention may comprise the following polymers as adhesive agents, for example: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC); microcrystalline cellulose, guar flour, alginic acid and/or alginates; synthetic polymers such as polyvinyl pyrrolidone, polyvinyl acetate (PVAC), polyvinyl alcohol (PVA), polymers of acrylic acid and their salts, polyacrylamide, polymethacrylates, vinyl pyrrolidone/vinyl acetate copolymers (such as Kollidon® VA64, BASF), polyalkylene glycols, such as polypropylene glycol or preferably polyethylene glycol, coblock polymers of polyethylene glycol, especially co-block polymers of polyethylene glycol and polypropylene glycol (Pluronic®, BASF), and mixtures of the polymers mentioned.

The adhesive agent used in the context of this invention may be a polymer which has a glass transition temperature (Tg) higher than 15° C., more preferably 40° C. to 150° C., especially 50° C. to 110° C.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Below the Tg, a polymer is usually glassy and hard, whereas above the Tg, it changes into a rubber-like to viscous state. The glass transition temperature is determined in the context of this invention by means of dynamic differential scanning calorimetry (DSC). For this purpose a Mettler Toledo DSC 1 apparatus, for example, can be used. The work is performed at a heating rate of 1-20° C./min, preferably 5-15° C./min, and at a cooling rate of 5-25, preferably 10-20° C./min.

Furthermore, the adhesive agent also includes solid, non-polymeric compounds which preferably contain polar side groups. Examples of these are sugar alcohols or disaccharides. Examples of suitable sugar alcohols and/or disaccharides are mannitol, sorbitol, xylitol, glucose, fructose, maltose and mixtures thereof. The term “sugar alcohols” in this context also includes monosaccharides. In particular, sorbitol and/or mannitol are used as adhesive agents.

Similarly, mixtures of the above-mentioned adhesive agents are possible.

In one particularly preferred embodiment, the adhesive agent is an agent containing or consisting of microcrystalline cellulose. In an alternative preferred embodiment, the adhesive agent is an agent containing or consisting of microcrystalline cellulose and sugar alcohol, especially an agent containing or consisting of microcrystalline cellulose and sorbitol and/or mannitol. The weight ratio of microcrystalline cellulose to sugar alcohol in this context is 1:5 to 5:1, preferably 1:1 to 3:1.

In preferred embodiments of the present invention, moxifloxacin and adhesive agent are used in an amount in which the weight ratio of moxifloxacin to adhesive agent is 10:1 to 1:10, more preferably 5:1 to 1:3, even more preferably 3:1 to 1:2, especially 2.5:1 to 1.5:1.

It is advantageous for the adhesive agent to be used in particulate form and for the adhesive agent to have a volume-average particle size (D50) of less than 500 μm, preferably 5 to 200 μm.

The expression “average particle diameter” or “volume-average particle size” relates in the context of this invention to the D50 value of the volume-average particle diameter determined by means of laser diffractometry. In particular, a Malvern Instruments Mastersizer 2000 was used to determine the diameter (wet measurement with ultrasound 60 sec., 2,000 rpm (preferably paraffin as dispersant), the evaluation being performed according to the Fraunhofer model). The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value. Analogously, the D90 value of the integral volume distribution is defined as the particle diameter at which 90% by volume of the particles have a smaller diameter than the diameter which corresponds to the D90 value.

In step (i) of the process of the invention, further pharmaceutical excipients may optionally be added to the mixture of moxifloxacin and adhesive agent. These are preferably the excipients described in more detail below (under process step (iv)). It is in principle possible in this connection for the excipients described to be added in step (i), in step (iv) or partly in step (i) and step (iv). In a preferred embodiment, a disintegrant may be added in step (i) and a flow-regulating agent and/or lubricant in step (iv).

In a preferred embodiment, in step (i) of the process of the invention,
(a) 20 to 80% by weight, more preferably 40 to 75% by weight, in particular 55 to 70% by weight moxifloxacin or pharmaceutically acceptable salts thereof and
(b) 20 to 80% by weight, more preferably 25 to 60% by weight, in particular 45 to 30% by weight adhesive agent,

based on the total weight of the mixture used, are blended. Where further pharmaceutical excipients are used together with the adhesive agent, the details specified above for component (b) refer to the weight of adhesive agent and further pharmaceutical excipients. In a preferred embodiment, the details specified component (b) refer to the weight of adhesive agent and disintegrant.

The mixing can be performed in conventional mixers. The mixing may, for example, be performed in compulsory mixers or free-fall mixers, e.g. using a Turbula T 10B (Bachofen AG, Switzerland). Alternatively, it is possible that the moxifloxacin is initially only mixed with part of the adhesive agent (e.g. 50 to 95%) before compacting (b), and that the remaining part of the excipients is added after the granulation step (c). In the case of multiple compacting, the excipients should preferably be mixed in before the first compacting step, between multiple compacting steps or after the last granulation step.

The moxifloxacin used in step (i) may have a volume-average particle size (D50) of, for example, more than 20 to 200 μm, preferably 50 to 150 μm.

The moxifloxacin used may alternatively be micronised. The micronisation is preferably performed before the compacting or before the moxifloxacin is blended with the excipients. Micronisation usually leads to an increase in the surface roughness. The micronisation is performed in, for example, pin mills or air impact mills. Micronisation may also by performed by wet grinding in ball mills. The micronised moxifloxacin preferably has a volume-average particle size (D(50)) of 0.5 to 20 μm, preferably 1 to 10 μm.

In step (ii) of the process of the invention, the mixture containing moxifloxacin and adhesive agent (and optionally further pharmaceutical excipients) from step (i) is compacted into a slug of the invention. Here, it is preferable that it is dry-compacting. Instead of the term “slug”, the expression “compacted material” is therefore also used in the context of this invention.

The compacting is preferably performed in the absence of solvents, especially the absence of organic solvents.

The compacting is preferably carried out in a roll granulator.

The rolling force is preferably 2 to 30 kN/cm, more preferably 5 to 15 kN/cm, especially 6 to 12 kN/cm.

The gap width of the roll granulator is, for example, 0.8 to 5 mm, preferably 2.0 to 4.5 mm, more preferably 3.0 to 4.0 mm, especially 3.2 to 3.8 mm.

The compacting apparatus used preferably has a cooling means. In particular, the process is conducted and, where applicable, cooled such that the temperature of the compacted material does not exceed 55° C.

The typical throughput through the compactor is usually 12-45 kg/h, preferably 15-30 kg/h. For this purpose, it is preferable for the above-mentioned gap width (especially 2.5 to 4.5 mm) and a roll width of 100 mm to be used.

The mixing conditions in step (i) and/or the compacting conditions in step (ii) are usually selected such that at least 10% of the surface of the resulting moxifloxacin particles is covered with adhesive agent, more preferably at least 20% of the surface, particularly preferably at least 50% of the surface, especially at least 60% of the surface.

The compacting conditions in step (ii) are generally selected such that the compacted material has an apparent density of 0.8 to 1.3 g/cm³, preferably 0.86 to 1.28 g/cm³, more preferably 0.92 to 1.21 g/cm³, especially 1.00 to 1.18 g/cm³.

The apparent density of the compacted material is calculated as follows:

apparent density_{compacted material}=mass_{compacted material}/volume_{compacted material}

Generally, there are two suitable methods for determining the apparent density, namely a) throughput method and b) punching method.

a) Throughput Method:

The throughput method is preferable especially when a roll compactor or roll granulator is used.

Establishing the Throughput Rate in Dry-Compacting:

Measurement is performed according to the weighing principle, in that the compacted mixture (=compacted material from step (ii)) are trapped and weighed precisely in a defined period under otherwise constant conditions. Any increase in the moisture of the compacted material that might occur is subsequently corrected by computation.

For this purpose—once the compactor is working at a constant roll speed, gap width and compacting force, i.e. after the start-up phase has passed over into the production phase—, the entire compacted material is caught, without loss, in a container suitable for pharmaceuticals, and the process time involved is recorded. This is done with a stop-watch, measuring a period of 2 minutes, or 120 sec., and the compacted material caught in that time is used for the measurement. After that, the compacted material is weighed and the moisture determined (halogen lamp moisture meter). The weighed material is corrected by the moisture difference before and after compacting, and then the mass flow is calculated by dividing the mass in kg by the time in minutes.

Result: throughput of compacted material in kg/min. By multiplying by 60, one obtains the throughput of compacted material in kg/h.

Measuring Principle of a Halogen Lamp Moisture Meter:

the measuring method is thermogravimetry, i.e. a defined mass is excited thermally and releases water where applicable. The change in weight is a measure of the moisture present (see, for example, Mettler-Toledo: Halogen Moisture Analyzer HG 63).

The apparent density is then calculated using the following formula:

apparent density=mass throughput/volume throughput; wherein

volume throughput=roll speed×roll width×roll diameter×π(pi)×gap width.

b) Punching Method:

The punching method is based on the principle of isolating and weighing a geometrically defined sample body from the compacted material.

Using a punching tool, a defined piece of flake is separated from the sample. The volume of that piece of compacted material corresponds to the punched volume.

A cylindrical punched piece is used. The volume of the punched piece Vpunch is defined as follows:

V_{punch}=π×r²×h

r is the radius of the punched piece, h is the height of the punched compacted material.

Regarding the apparent density of the compacted material, the relationship

apparent density_{compacted material}=mass_{punch}/volume_{punch}

applies analogously. The punching method b) is preferably always used whenever the throughput method a) cannot be employed, because of the geometry of the device in process step (ii).

The compacted material resulting in process step (ii) can also be characterised by its porosity. It usually has a porosity of between 0.16 and 0.45, preferably between 0.25 and 0.43, particularly preferably between 0.28 and 0.40.

The typical throughput through the compactor is usually 12-45 kg/h, preferably 15-30 kg/h. For this purpose, it is preferable for the above-mentioned gap width (especially 2.5 to 4.5 mm) and a roll width of 100 mm to be used.

The porosity is calculated according to the formula:

porosity epsilon=(1−(true density of starting material/apparent density of compacted material)

The starting material is the mixture obtained in process step (i). The true density can be determined with a gas pycnometer. The gas pycnometer is preferably a helium pycnometer; in particular, the AccuPyc 1340 helium pycnometer from the manufacturer Micromeritics, Germany, is used.

Example of a Calculation of the Porosity:

| True density | 1.4 | g/cm³ | 1.6 | g/cm³ | 1.4 | g/cm³ | 1.4 | g/cm³ |
|---|---|---|---|---|---|---|---|---|
| starting | | | | | | | | |
| material | | | | | | | | |
| Throughput | 15 | kg/h | 15 | kg/h | 45 | kg/h | 15 | kg/h |
| Gap width | 3.24 | mm | 3.24 | mm | 3.24 | mm | 5.00 | mm |
| Roll speed | 1/min | | 1/min | | 3/min | | 1/min | |
| Roll diameter | 250 | mm | 250 | mm | 250 | mm | 250 | mm |
| Roll width | 100 | mm | 100 | mm | 100 | mm | 100 | mm |

Results:

| | | | | | | | | |
|---|---|---|---|---|---|---|---|---|
| Apparent | 0.982 | g/cm³ | 0.982 | g/cm³ | 0.982 | g/cm³ | 0.637 | g/cm³ |

| | | | | |
|---|---|---|---|---|
| density | | | | |
| Porosity | 0.298 | 0.386 | 0.298 | 0.545 |

In step (iii) of the process of the invention the compacted material is granulated. The granulating can be performed using processes known in the state of the art.

In a preferred embodiment, the granulation conditions are selected such that the resulting particles (granules) have a volume-average particle size (D50 value) of 20 to 600 μm, more preferably 50 to 400 μm, even more preferably 80 to 200 μm, especially 90 to 130 μm. The D90 value of the resulting particles (granules) is usually 500 to 1,300 μm, preferably 800 to 1,200 μm.

In an alternative preferred embodiment, the D50 value is 30 μm to 200 μm and/or the D90 value is 250 μm to 1,200 μm.

In addition, the granulation conditions are preferably selected such that the resulting granules have a bulk density of 0.3 to 0.85 g/ml, more preferably 0.4 to 0.8 g/ml, especially 0.5 to 0.7 g/ml. The Hausner factor is usually in the range from 1.02 to 1.4, more preferably from 1.04 to 1.20 and especially from 1.1 to 1.25. The “Hausner factor” in this context means the ratio of tapped density to bulk density.

The volume-average particle size (D50) of the final mixture ready for tableting is preferably 20-300 μm, particularly preferably 45-100 μm. The D90 value with this particle size distribution is preferably 350-1,500 μm, particularly preferably 500-1,000 μm.

In a preferred embodiment, the granulation is performed by means of a granulator screen, which may be integrated in the compactor or separate; or in a different screen mill. In this case, the mesh width of the screen insert is usually 0.1 to 4 mm, preferably 0.5 to 2 mm, more preferably 0.8 to 2 mm, especially 1.0 to 1.5 mm.

It may happen that the moxifloxacin adhesive agent particles do not have a sufficiently rough surface, so that the compacting step (ii) described above is rendered more difficult. Therefore, depending on the nature of the surface, the compacting step (ii) and the granulating step (iii) can be repeated if need be.

In a further embodiment, the process of the invention is therefore adapted such that multiple compacting occurs, with the granules resulting from step (iii) being returned one or more times to the compacting (ii).

The granules from step (iii) are preferably returned 1 to 5 times, especially 2 to 3 times.

In the case of multiple compacting, the granulating (iii) is preferably performed with a Frewitt screen. Screening is preferably performed with screen diameters of 50 to 250 μm.

In the case of multiple compacting, it is also possible for only parts of the amounts of excipients specified above to be added in step (i), with the remaining parts added before the further compacting processes.

The above statements regarding steps (i) and (ii) apply not only to the production of the tablets of the invention, but also to the production of the compacted material of the invention. The above statements regarding (i) to (iii) also apply analogously to the production of the granules of the invention.

In step (iv) of the process of the invention, the granules obtained in step (iii) are pressed into tablets, i.e. the step involves compression into tablets. The compression may be performed with tableting machines known in the state of the art. Process step (iv) is preferably performed in the absence of solvents, especially organic solvents i.e. as dry compression. Examples of suitable tableting machines are eccentric presses or rotary presses. As an example, a Fette® 102i (Fette GmbH, Germany) can be used. In the case of rotary presses and eccentric presses, a compressive force of 2 to 40 kN, preferably 2.5 to 35 kN, is usually applied. With eccentric presses, compressive forces of up to 100 kN are also possible, however.

In step (iv) of the process of the invention, excipients may be added to the granules from step (iii).

Examples of suitable excipients are additives to improve the powder flowability (e.g. disperse silica), tablet lubricants (e.g. talcum, stearic acid, adipic acid, sodium stearyl fumarate and/or magnesium stearate) and disintegrants. In addition, the adhesive agents mentioned under step (i) may also be added. In particular, flow-regulating agents and/or lubricants are added in step (iv). If not already added in step (i), disintegrant is preferably also added in step (iv).

“Disintegrants” is the term generally used for substances which accelerate the disintegration of a dosage form, especially a tablet, after it is placed in water. Suitable disintegrants are, for example, organic disintegrants such as carrageenan, croscarmellose and crospovidone.

Alternatively, alkaline disintegrants can be used. The term “alkaline disintegrants” means disintegrants which, when dissolved in water, produce a pH level of more than 7.0. More preferably, inorganic alkaline disintegrants are used, especially salts of alkali and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

Disintegrants are normally used in an amount of 0.5 to 15% by weight, preferably 3 to 7% by weight, based on the total weight of the formulation.

One example of an additive to improve the powder flowability (flow-regulating agent) is disperse silica, e.g. known under the trade name Aerosil®. Preferably, silica is used with a specific surface area of 50 to 400 m²/g, determined by gas adsorption in accordance with Ph. Eur., 6th edition, 2.9.26. The task of flow-regulating agents is usually to reduce both the friction (cohesion) between the individual particles of powder or granules and also their adherence to the wall surfaces of the press apparatus.

Additives to improve the powder flowability are generally used in an amount of 0.1 to 3% by weight, based on the total weight of the formulation.

In addition, lubricants may be used. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate and/or magnesium stearate.

Lubricants are generally used in an amount of 0.1 to 3% by weight, based on the total weight of the formulation.

The amounts of excipients added in step (iv) usually depend on the type of tablet to be produced and the amount of excipients which were already added in steps (i) or (ii).

The ratio of active agent to excipients is preferably selected such that the resulting tablets contain
(a) 35 to 85% by weight, more preferably 45 to 75% by weight, in particular 55 to 65% by weight moxifloxacin or its pharmaceutically acceptable salts, and
(ii) 15 to 65% by weight, more preferably 25 to 55% by weight, especially 35 to 45% by weight pharmaceutically acceptable excipients, based on the total weight the non-film-coated tablet.

These amounts specified are especially preferable if the process of the invention is used to produce tablets which are to be swallowed unchewed.

The tablets produced by the process of the invention may therefore be tablets which can be swallowed unchewed (non-film-coated or preferably film-coated). They may likewise be chewable tablets or dispersible tablets. “Dispersible tablet” here means a tablet to be used for producing an aqueous suspension for swallowing.

Furthermore, the tableting conditions in the process of the invention are preferably selected such that the resulting tablets have a ratio of tablet height to weight of 0.005 to 0.3 mm/mg, particularly preferably 0.05 to 0.01 mm/mg.

In addition, the resulting tablets preferably have a breaking strength of 160 to 400 N, particularly preferably 200 to 350 N especially 220 to 270 N. The breaking strength is determined in accordance with Ph. Eur. 6th main edition 2008, section 2.9.8.

In addition, the resulting tablets preferably have a friability of less than 2%, particularly preferably less than 1%, especially less than 0.5%. The friability is determined in accordance with Ph. Eur. 6.0, section 2.9.7.

Finally, the tablets of the invention usually have a “content uniformity” of 95 to 105% of the average content, preferably 97 to 103%, especially 99 to 101%. The “content uniformity” is determined in accordance with Ph. Eur. 6.0, section 2.9.6.

In the case of an IR formulation, the release profile of the tablets of the invention after 10 minutes according to the USP method (preferably the “paddle” method) usually indicates a content released of at least 30%, preferably at least 60%, especially at least 80%.

The above details regarding breaking strength, friability, content uniformity and release profile preferably relate here to the non-film-coated tablet.

In a preferred embodiment, no lactose or at least only small amounts are added in the process of the invention. Despite the absence of lactose, the desired breaking strength is achieved. One subject matter of the invention is therefore a tablet comprising moxifloxacin, containing less than 10% by weight lactose, preferably less than 5% by weight lactose, which in particular is substantially free of lactose, wherein the tablet has a breaking strength of 140 to 400 N.

In the optional step (v) of the method of the invention, the tablets from step (iv) are film-coated. For this purpose, the methods of film-coating tablets which are standard in the state of the art may be employed.

For film-coating, macromolecular substances are preferably used, such as modified celluloses, polymethacrylates, polyvinyl pyrrolidone, polyvinyl acetate phthalate, zein and/or shellack.

The thickness of the coating is preferably 10 to 100 μm, more preferably 15 to 50 μm.

The process of the invention is in particular suitable for the preparation of tablets containing a large amount of moxifloxacin or pharmaceutically acceptable salts thereof. In a preferred embodiment, the tablets of the invention contain 100 to 1,000 mg, particularly preferably 200 to 800 mg, especially 200 to 600 mg moxifloxacin or pharmaceutically acceptable salts thereof.

It has become clear that the process of the invention can preferably be carried out with the following amounts of substances:

200 to 600 mg moxifloxacin, preferably 380 to 450 mg moxifloxacin, wherein moxifloxacin is preferably used in the form of the free base or the hydrochloride;

100 to 300 mg adhesive agent, preferably 180 to 250 mg adhesive agent, wherein microcrystalline cellulose is preferably used as the adhesive agent;

20 to 50 mg, preferably 28 to 38 mg disintegrant, wherein croscarmellose sodium is preferably used as the disintegrant;

0 to 10 mg, preferably 3 to 8 mg flow-regulating agent, wherein disperse silica (Aerosil®) is preferably used as the flow-regulating agent;

0 to 20 mg, preferably 5 to 15 mg lubricant, wherein magnesium stearate is preferably used as the lubricant;

The process of the invention is therefore preferably carried out with the above-mentioned substances. One subject matter of the invention is therefore also tablets obtainable by the process of the invention and containing the above-mentioned formulation.

As explained above, the subject matter of the invention is thus not only the process of the invention, but also the tablets produced with that process. It has been found that the tablets produced with this process may have a bimodal pore size distribution. Hence, the subject matter the invention comprises tablets containing moxifloxacin or pharmaceutically acceptable salts thereof, and optionally pharmaceutically acceptable excipients, wherein the tablets have a bimodal pore size distribution.

This tablet of the invention is formed when the granules from process step (iii) are compressed. This compressed material consists of solid material and pores. The pore structure can be characterised more specifically by determining the pore size distribution.

The pore size distribution was determined by means of mercury porosimetry. Mercury porosimetry measurements were made with the Micromeritics, Norcross, USA, “Pore-sizer” porosimeter. The pore sizes were calculated assuming a mercury surface tension of 485 mN/m. The cumulative pore volume was used to calculate the pore size distribution as the cumulative frequency distribution or proportion of the pore fractions in per cent. The average pore diameter (4V/A) was determined from the total specific mercury intrusion volume (Vgesᵢₙₜ) and the total pore surface area (Agespₚₒᵣ) according to the following equation.

$$4 V / A = \frac{4 \cdot {Vges}_{int} [ ml / g ]}{{Ages}_{por} [ {m}^{2} / g ]}$$

“Bimodal pore size distribution” is understood to mean that the pore size distribution has two maxima.

The invention will now be explained with reference to the following examples.

### EXAMPLES

### Examples 1 to 3 in Accordance with the Invention

A mixture of moxifloxacin hydrochloride and adhesive agent was prepared by intensively mixing moxifloxacin and adhesive agent together with croscarmellose Na for 10 min. with a free-fall mixer. After that, the mixture was crushed on a roll compactor suitable for pharmaceuticals, with a gap width of 3.5 mm and across a crusher screen with a mesh width of 1.25 mm. The crushed compacted material obtained (=granules) was mixed with highly disperse silica after screening (free-fall mixer drum hoop type) and finally mixed with magnesium stearate (free-fall mixer drum hoop type). After pressing into tablets of a given size on a high-performance rotary tablet press, the standard in-process checks for the dosage form were carried out.

The amounts used can be seen from Table 1.

### Examples 1a to 3a in Accordance with the Invention

The tablet cores according to Examples 1 to 5 were film-coated. For this purpose, hypromellose (Pharmacoat 603) and polyethylene glycol 6,000 were mixed with water, and, after they had dissolved, mixed with a separately prepared suspension of titanium dioxide and iron oxide in water. The tablet cores were coated with the resulting suspension in a perforated-drum coater. The amounts used can be seen from Table 1.

Alternatively, ready-made coats and different proportions of hypromellose (weight-average molecular weight 10,000-150,000), polyethylene glycol (weight-average molecular weight 200 to 8,000), titanium dioxide and dye pigments may also be used.

### Examples 4, 4a, 5 and 5a in Accordance with the Invention

Analogously to Examples 1-3 and 1a-3a, Examples 4-5 and 4a-5a were prepared, wherein, instead of the hydrochloride salt, moxifloxacin was used in the form of the free base.

The amounts used can be seen from Table 2.

### Comparative Example 6

A tablet core as described in Example 6 of EP 1 128 831 B1 was prepared by wet granulation. The amounts used can be seen from Table 3.

Comparison of the Physical Properties

The physical properties of the resulting tablets are compared in Table 4

It can be seen that the tablets prepared by (dry)-compacting (both film-coated and non-film-coated) exhibit advantageous properties with regard to breaking strength and friability. The tablets of the invention are also more suitable for lacquer coating; less spalling occurs.

It is also clear that lactose-free formulations exhibit better content uniformity.

To sum up, it can be stated that with the process of the invention, the physical properties of the resulting tablets could be positively influenced. Moreover, the process of the invention is advantageous from the point of view of reducing the energy requirements, which are not ideal in the process of the state of the art, because the moisture first has to be incorporated in the form of a granulation solution and then removed again in a complex drying process.

TABLE 1

| | Example 1 | % by | Example 2 | % by | Example 3 | % by |
|---|---|---|---|---|---|---|
| Core | mg | weight | mg | weight | mg | weight |
| Moxifloxacin HCl | 436.80 | 64.35 | 436.80 | 64.35 | 436.80 | 64.35 |
| MCC (microcr. | 195.00 | 28.73 | 128.00 | 18.86 | 136.00 | 20.04 |
| cellulose) | | | | | | |
| Lactose- | 0.00 | 0.00 | 0.00 | 0.00 | 68.00 | 10.02 |
| monohydrate | | | | | | |
| Mannitol | 0.00 | 0.00 | 67.00 | 9.87 | 0.00 | 0.00 |
| Croscarmellose Na | 32.00 | 4.71 | 32.00 | 4.71 | 32.00 | 4.71 |
| Mg stearate (E572) | 10.00 | 1.47 | 10.00 | 1.47 | 6.00 | 0.88 |
| Aerosil | 5.00 | 0.74 | 5.00 | 0.74 | 0.00 | 0.00 |
| | 678.80 | 100.00 | 678.80 | 100.00 | 678.80 | 100.00 |
| Film | Example 1a | | Example 2a | | Example 3a | |
| Hypromellose | 10.80 | | 10.80 | | 10.80 | |
| Titanium dioxide | 3.24 | | 3.24 | | 3.24 | |
| Polyethylene glycol | 3.60 | | 3.60 | | 3.60 | |
| Iron oxide, red | 0.36 | | 0.36 | | 0.36 | |
| | 18.00 | | 18.00 | | 18.00 | |
| Film-coated | 696.80 | | 696.80 | | 696.80 | |
| tablet (FT) | | | | | | |
| Production process | dry | | dry | | dry | |
| | granulation | | granulation | | granulation | |

TABLE 2

| | Example 4 | | Example 5 | |
|---|---|---|---|---|
| 1st core | mg | % by weight | mg | % by weight |
| Moxifloxacin (free base) | 400.00 | 58.93 | 400.00 | 58.93 |
| MCC (microcr. cellulose) | 231.80 | 34.15 | 230.80 | 34.00 |
| Croscarmellose Na | 32.00 | 4.71 | 5.00 | 0.74 |
| Aerosil | 5.00 | 0.74 | 5.00 | 0.74 |
| Kollidon VA 64 | 0.00 | 0.00 | 28.00 | 4.12 |
| Mg stearate (E572) | 10.00 | 1.47 | 10.00 | 1.47 |
| | 678.80 | 100.00 | 678.80 | 100.00 |
| Film | Example 4a | | Example 5a | |
| Hypromellose | 10.80 | | 10.80 | |
| Titanium dioxide | 3.24 | | 3.24 | |
| Polyethylene glycol | 3.60 | | 3.60 | |
| Iron oxide, red | 0.36 | | 0.36 | |
| | 18.00 | | 18.00 | |
| Film-coated tablet (FT) | 696.80 | | 696.80 | |
| Production process | dry granulation | | dry granulation | |

TABLE 3

| | Example 6 | |
|---|---|---|
| Core | (comparative) | % by weight |
| Moxifloxacin HCl | 436.80 | 64.35 |
| MCC (E460 (l)) | 136.00 | 20.04 |
| Lactose-monohydrate | 68.00 | 10.02 |
| Mannitol | 0.00 | 0.00 |
| Crosscarmellose Na | 32.00 | 4.71 |
| Mg stearate (E572) | 6.00 | 0.88 |
| Aerosil | 0.00 | 0.00 |
| | 678.80 | 100.00 |
| Film | no film-coated | |
| | tablets produced | |
| Production process | aqueous granulation | |

TABLE 4

| | Example 6 | | | | | |
|---|---|---|---|---|---|---|
| | (comparative) | Example 1 | Example 2 | Example 3 | Example 4 | Example 5 |
| Compressive | | | | | | |
| force [kN] | | | | | | |
| VPK | 14 | 12 | 12 | 12 | 8 | 8 |
| HPK | 23 | 23 | 23 | 23 | 15 | 15 |
| srel [%] | 4.3 | 5.5 | 4.2-6.8 | 8.6 | 7.22 | 4.78 |
| BFK cores [N]/ | 152.66/7.42 | 250.72/7.17 | 230.43/5.94 | 235.6/5.36 | 188/7.32 | 200/5.63 |
| srel. [%] | | | | | | |
| Core abrasion [%] | capped; >1% | 0.26 | 0.25 | 0.31 | 0.55 | 0.41 |
| Spalls [%] | 1 | 0 | 0 | 0 | 0 | 0 |
| BFK FT [N]/ | not capable | 313.13/6.13 | 285.6/6.77 | 290.03/8.95 | 271.73 | 284 |
| srel. [%] | of lacquer | | | | | |
| | coating | | | | | |
| Disintegration | no FT | 1′43″ | 2′29″ | 2′25″ | 1′25″ | 4′07″ |
| FT [min] | produced | | | | | |

BFK = breaking strength
VPK = precompressive force
HPK = main compressive force
srel = relative standard deviation
FT = film-coated tablet
Core = non-film-coated tablet

1-16. (canceled)

17. A process for the preparation of tablets comprising moxifloxacin, the process comprising the steps of:
(i) providing 200 to 600 mg moxifloxacin or pharmaceutically acceptable salts thereof mixed with an adhesive agent, wherein the adhesive agent comprises microcrystalline cellulose;
(ii) compacting into a slug;
(iii) granulating the slug; and
(iv) compressing the resulting granules into tablets, optionally with the addition of further pharmaceutical excipients.

18. The process according to claim 17, wherein the used moxifloxacin is moxifloxacin hydrochloride.

19. The process according to claim 17, wherein the used moxifloxacin comprises 0.1 to 5 wt % water.

20. The process according to claim 17, wherein the weight ratio of moxifloxacin to adhesive agent is 3:1 to 1:2.

21. The process according to claim 17, wherein the adhesive agent contains a mixture of microcrystalline cellulose and mannitol.

22. The process according to claim 21, wherein the weight ratio of microcrystalline cellulose to mannitol is 1:5 to 5:1.

23. The process according to claim 17, wherein the further excipients comprise disintegrants, flow-regulating agents and/or lubricants.

24. The process according to claim 23, wherein the flow-regulating agent is disperse silicon dioxide.

25. The process according to any one of claims 23, wherein the lubricant is talc, stearic acid, adipic acid, sodium stearyl fumarate and/or magnesium stearate.

26. The process according to claim 17, wherein the tablet of step (iv) is film-coated in a step (v).

27. The process according to claim 26, wherein the thickness of the coating is 10 to 100 μm.

28. The process according to claim 17, wherein the final mixture which is to be compressed into a tablet in step (iv) has a mean volume particle size D50 of 20 to 300 μm.

29. A tablet comprising moxifloxacin or pharmaceutically acceptable salts thereof obtainable by a process according to claim 17, wherein the tablet is substantially free of lactose and has a breaking strength of 160 to 300 N.

30. The tablet according to claim 29, wherein the tablet has a friability of less than 1% and a content uniformity of 95 to 105%.

31. A slug comprising moxifloxacin, obtainable by a process comprising the steps of
(i) providing 200 to 600 mg moxifloxacin or pharmaceutically acceptable salts thereof mixed with an adhesive agent, wherein the adhesive agent contains microcrystalline cellulose; and
(ii) compacting into a slug.
